(19) United States
(12) Patent Application Publication
(10) Pub. No.: US 2009/0259220 A1
(43) Pub. Date: Oct. 15, 2009

(54) Treatment Devices and Methods

(75) Inventors: William M. Appling, Granville, NY (US); Robert M. Pearson, San Jose, CA (US); Kevin Moss, Tracy, CA (US); Shaily Bhargav, Mountain View, CA (US); David A. Blau, Chico, CA (US)

Correspondence Address: ANGIODYNAMICS, INC., 603 QUEENSBURY AVENUE, QUEENSBURY, NY, 12804, US

(73) Assignee: AngioDynamics, Inc., Queensbury, NY (US)

(21) Appl. No.: 12/421,302

(22) Filed: Apr. 9, 2009

Related U.S. Application Data
(60) Provisional application No. 61/043,728, filed on Apr. 9, 2008.

(51) Int. Cl. A61B 18/20

(52) U.S. Cl. 606/10

(57) ABSTRACT

Systems disclosed herein contain a source for providing one or more energy sources as well as substances singly or in combination, and a kit containing a delivery device. The delivery device is configured to be releasably coupled to the source for delivering the energy as well as the substances to a location remote from the source which may include delivery for a patient.

### RELATED APPLICATIONS

This application claims priority under 35 U.S.C. 119(e) to U.S. Provisional Application No. 61/043,728, filed Apr. 9, 2008, which is incorporated herein by reference.

### BACKGROUND OF THE INVENTION

### Field of the Invention and Description of the Related Art

The present description relates in general to systems that contain a source for providing energy as well as substances singly as well as in combination, and a kit containing a delivery device. The delivery device is configured to be releasably coupled to the source for delivering the one or more energies as well as substances to a location remote from the source. In particular, the present description relates to a laser system that contains a laser source for providing laser radiation, and a kit containing a laser delivery device. The laser delivery device contains one or more optical fibers, and may be releasably coupled to the laser source for delivering laser radiation to a location on or in a patient.

### BRIEF SUMMARY OF THE INVENTION

This application discloses a kit comprising a treatment delivery device and a product key separate or detachable from the treatment delivery device prior to its use, wherein the treatment delivery device is configured for coupling with a treatment source, and where the product key is electromagnetically readable by the treatment source.

This application also discloses a kit comprising a laser delivery device and an electromagnetically readable product key, wherein the product key is physically separated from the laser delivery device.

This application further discloses a treatment delivery device, comprising a circuit powered by direct current, wherein the circuit comprises direct current power storage and data storage, and the data storage comprises a product key unique to the treatment delivery device.

This application further discloses a laser delivery device, comprising a proximal connector, wherein the proximal connect comprises a direct current (DC) circuit, the DC circuit comprises a mutable data storage, and the mutable data storage comprises a product key unique to the laser delivery device.

This application further discloses a hardware piece, comprising a DC circuit, wherein the DC circuit comprises a DC power storage and a mutable data storage, and the mutable data storage comprises an algorithm for limiting usage of a treatment delivery device. The hardware piece may be a door interlock configured to be releasably coupled to a treatment delivery device, such as a laser delivery device.

Throughout the present disclosure in its entirety, any and all of the one, two, or more features disclosed herein following the term “example” may be practiced in any combinations of two, three, or more thereof, whenever and wherever appropriate as understood by one of ordinary skill in the art. Some of these examples are themselves sufficient for practice without being combined with any other features, as understood by one of ordinary skill in the art. Throughout the present disclosure in its entirety, any and all of the descriptions following the term “example” are for illustration only, without limiting the scope of any of the referenced terms or phrases either within the context or outside the context of such descriptions.

### BRIEF DESCRIPTION OF THE SEVERAL VIEWS OF THE DRAWINGS

FIG. 1 illustrates a kit according to the present application.

FIG. 2 illustrates a treatment system according to the present application.

FIG. 3 illustrates the coupling of a reader and a treatment source according to the present application.

FIG. 4 illustrates another coupling of a reader and a treatment source according to the present application.

FIG. 5 illustrates the coupling of a treatment delivery device and a treatment source according to the present application.

FIG. 6 illustrates a connector according to the present application.

FIG. 7 illustrates another connector according to the present application.

FIG. 8 illustrates a schematic of a DC circuit according to the present application.

FIG. 9 illustrates another schematic of a DC circuit according to the present application.

### DETAILED DESCRIPTION OF THE INVENTION

The present description relates in general to systems that contain a source for providing energy as well as substances singly as well as in combination, and a kit containing a delivery device. The delivery device is configured to be releasably coupled to the source for delivering energy as well as substances singly as well as in combination to a location remote from the source. In particular, the present description relates to a laser system that contains a laser source for providing laser radiation, and a kit containing a laser delivery device. The laser delivery device contains one or more optical fibers, and may be releasably coupled to the laser source for delivering laser radiation to a location on or in a patient.

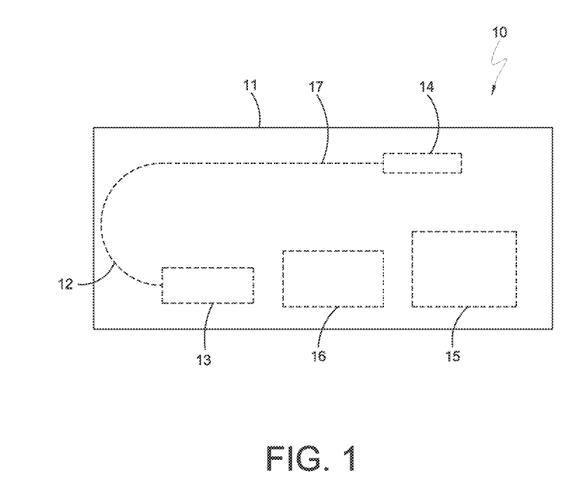

In certain embodiments, as illustrated in FIG. 1, a kit 10 contains a laser delivery device 12 and a unique product key (not shown) capable of identifying the supplier of laser delivery device 12 or kit 10. The product key may further encode one or more of model number, device type, serial number, date of manufacturer, expiration date, and other general or unique product information. The product key may be encrypted digitally or in other methods known in the art. Laser delivery device 12 contains a distal portion 14, a body 17, and a proximal connector 13. Body 17 may include one or more optical fibers. Connector 3 may include one or more optical fiber connectors, such as sub miniature A (SMA), sub miniature C (SMC), enterprise systems connection (ESCON), ferrule connector (FC), fiber distributed data interface (FDDI), local connector (LC), mechanical transfer (MT), mechanical transfer registered jack (MT-RJ), MU, subscriber connector (SC), straight tip/bayonet fiber optic connector (ST/BFOC), Toshiba link (TOSLINK), biconic, D4, E2000, media interface connector (MIC), multi-fiber push on (MPO), MTP, or opti-jack. Product key is not disposed on or in laser delivery device 12. Rather, the product key is included on or in packaging 11 of kit 10, on or in an insert 16 included in kit 10, or otherwise detached or detachable from laser delivery device 12. Insert 16 may be an instruction for use (IFU), or a stand-alone member (e.g., a card such as a swipe card, a flag (e.g., one that is detachable from device 12), a ticket, a member detachably coupled to device 12 but is not a functional component of device 12 (e.g., a cap that covers connector 13 or distal end of distal portion 14, which would be removed prior to use of device 12). For example, the product key may be printed, encoded, embedded, or otherwise stored on or in packaging 11 or insert 16. The product key may be in the form of a two-dimensional pattern (e.g., a bar code, a pixel pattern), an AC or RF tag or transponder, a magnetic member (e.g., a strip, a chip), an optical member (e.g., a hologram, an optical strip or chip or disk), or other data storage form or medium.

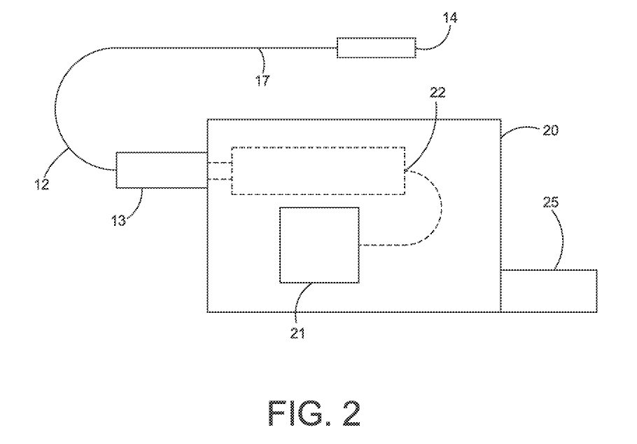

As illustrated in FIG. 2, connector 13 may be releasably coupled to a laser source 20. Laser source 20 may contain therein a laser generation circuit 22. Laser generation circuit 22 may include one or more of: a processor (e.g., a controller), a DC power storage (e.g., a capacitor, a battery), a DC power management circuit, a time keeper (e.g., an oscillator), a laser with an on and off sensing circuit, an input signal sensing circuit, or a data storage (e.g., non-volatile memory). A reader 21 is at least one of electrically, optically, as well as mechanically (such as a set-top box or A side attachment), coupled to laser source 20, such as to laser generation circuit 22 therein. Reader 21 is configured to read the product key. An interlock 25 may be connected to laser source 20 to enable the activation of laser generation circuit 22; disconnecting interlock 25 from laser source 20 would disable laser generation circuit 22.

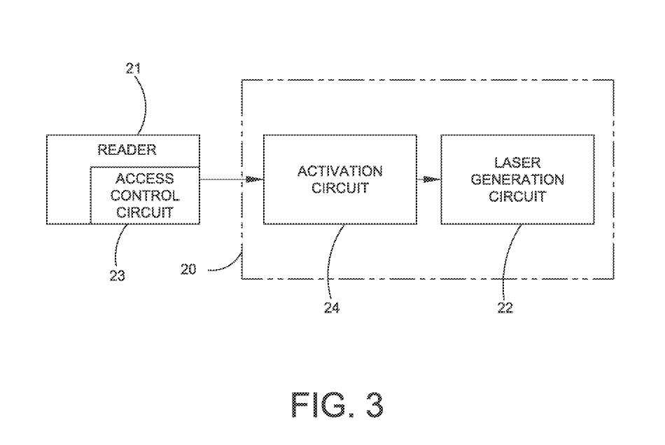

As illustrated in FIG. 3, reader 21 may contain an access control circuit 23 that receives the reading result on the product key and pass it onto an activation circuit 24 in laser source 20. Activation circuit 24 is configured to activate laser generation circuit 22 in laser source 20 when the product key is authenticated by reader 21, and any other requisites are satisfied (e.g., interlock 25 as illustrated in FIG. 2 is properly connected to laser source 20).

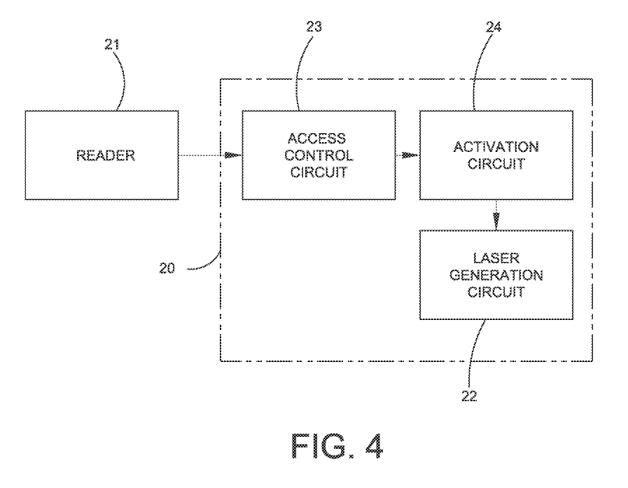

In alternative embodiments, as illustrated in FIG. 4, access control circuit 23 may be included within laser source 20. In further alternative embodiments (not shown), reader 20 may be integrated, embedded, or otherwise included in laser 20. In further alternative embodiments (not shown), access control circuit 23 may be a portion of activation circuit 24. In further alternative embodiments (not shown), activation circuit 24 may be a portion of access control circuit 23. The interworking among reader 21, access control circuit 23, activation circuit 24, and laser generation circuit 22 as described herein above applies to all these and other alternative embodiments.

It is noted that communication (e.g., data transfer) between reader 21 and laser device 20 or activation circuit 24 therein may be bi-directional or two-way. It is further noted that communication (e.g., data transfer) between reader 21 and the data storage that carries the product key on or in kit 10 may also be bi-directional or two-way. Such bi-directional or two-way communication applies at least to the configurations depicted in FIGS. 3-4.

Access control circuit 23 may include one or more of: a processor (e.g., a controller), a DC power storage (e.g., a capacitor, a battery), a DC power management circuit, a time keeper (e.g., an oscillator), a laser with an on and off sensing circuit, an input signal sensing circuit, or a data storage (e.g., non-volatile memory). Activation circuit 24 may include one or more of a processor (e.g., a controller), a DC power storage (e.g., a capacitor, a battery), a DC power management circuit, a time keeper (e.g., an oscillator), a laser with on and off sensing circuit, an input signal sensing circuit, or a data storage (e.g., non-volatile memory). Reader 21 may be a pattern recognition device (e.g., barcode reader), an RFID reader, a magnetic reader, an optical reader, or other suitable information recognition devices.

Referring now to FIGS. 1-4, in operation, the user may present the product key or kit 10 or a subcomponent thereof containing the product key to reader 21 for authentication. Upon receiving the product key, Reader 21 may then determine whether or not the product key satisfies an authentication algorithm pre-loaded into a data storage medium in reader 21 or laser source 20. The authentication algorithm may be configured for at least recognizing kit 10 singly or in combination with device 12 as being provided by a selected supplier (e.g., manufacturer, distributer). The authentication algorithm may first run a decryption subroutine if the product key is encrypted. If the product key can be properly decrypted and satisfies the pre-loaded authentication algorithm, then laser delivery device 12 is deemed authenticated as being provided by the selected supplier. Reader 21 may signal access control circuit 23 to allow activation circuit 24 to activate laser generation circuit 22 in laser source 20. When interlock 25 and laser delivery device 12 are properly connected to laser source 20, laser generation circuit 24 can be activated to provide a laser beam that is transmitted through body 17 of laser delivery device 12 and emitted out of distal portion 14. If reader 21 fails to authenticate the product key, laser generation circuit 24 would not be activated even when interlock 25 and laser delivery device 12 are properly coupled to laser source 20.

Besides the product key authentication algorithm, the data storage medium in reader 21 or laser source 20 may further store a database of product keys that reader 21 has ever read (optionally including the date and time stamps, locations, event logs, error logs, and other related information of such prior readings), as well as a database of product keys that are pre-authorized by the selected supplier. In alternative embodiments, the data storage medium may establish a communication with a remote-site (e.g., a computer or server through network or internet) on which the database(s) of product keys reside. The product key received by reader 21 may further be compared (e.g., by using a pre-loaded comparison algorithm) against the product keys store in the database(s) to determine if the received product key has ever been read by reader 21 as well as by other reader(s) 21 known to the selected supplier. If not, then laser delivery device 12 may be deemed as a brand new device that warrants proper authentication. Any and all information related to the reading (e.g., date and time, location, reading result, event log, error log, and other related information) and the successful authentication may be recorded on the data storage medium in reader 21 or laser source 20, as well as through the established communication at the remote site, if present, for record-keeping, as well as for future reference.

If the product key received by reader 21 is determined to have been read before according to the database(s), laser delivery device 12 may be deemed as used. In certain embodiments, the product key authentication algorithm may be programmed such that any product keys deemed as used fails the authentication process. As such the corresponding laser delivery device 12 may be rendered as a single-use disposable, and cannot be reused at all. In alternative embodiments, the product key authentication algorithm may be programmed such that certain or all product keys as deemed used may be granted a finite number (e.g., 2, 3, 4 or more) of reuse. If the received product key is deemed as used but within the reuse limit according to the database(s) in reader 21 or laser source 23 or at the remote site, then authentication is granted. If the received product key is deemed to have exceeded its reused limit, then the product key is deemed expired, and authentication is denied. As such, the corresponding laser delivery device 12 may be rendered as a multi-use disposable. Any and all information related to the reading (e.g., date and time, location, reading result, event log, error log, and other related information) and the successful authentication may be recorded on the data storage medium in reader 21 or laser source 20, as well as through the established communication at the remote site, if present, for record-keeping, as well as for future reference.

Reader 21 as well as laser source 20 may be configured to report the success or failure of product key authentication to the user. The report may be visual (e.g., print out, signal light, alphanumeric display), auditory (e.g., beeps, ring tones, simulated speech, prior recordings), as well as tactile (e.g., vibration). Either the product key resides on a mutable data storage, or at least one of reader 21 or access control circuit 23 contains a mutable data storage, or both.

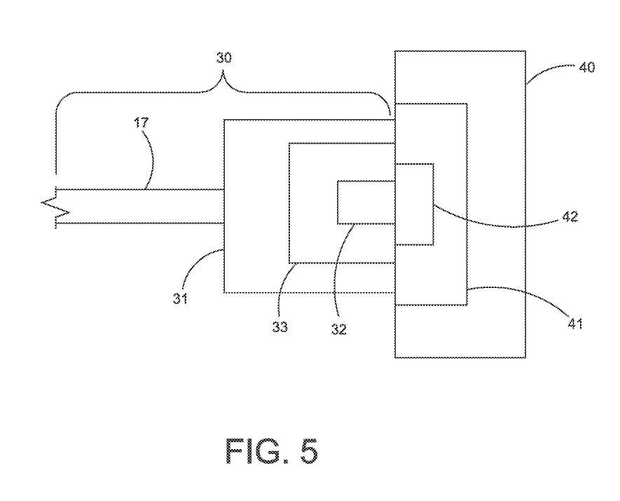

In certain embodiments, as illustrated in FIG. 5, connector 31 of distal portion 30 of a laser delivery device is configured to be releasably coupled to laser source 40. Connector 31 may contain an optical connector 32, and a direct current (DC) circuit 33. Optical connect 32 may be a component of DC circuit 33. Outer surface of connector 30 or a portion thereof may also be a component of DC circuit 33. When connector 31 is coupled to laser source 40, optical connector 32 may be at least optically coupled to a laser generation circuit 42, and DC circuit 33 may be at least electrically coupled to an access control circuit 41.

DC circuit 33 may contain one or more of: a processor (e.g., a controller), a DC power storage (e.g., a capacitor, a battery), a DC power management circuit, a time keeper (e.g., an oscillator), a laser with on and off sensing circuit, an input signal sensing circuit, or a data storage (e.g., non-volatile memory). DC circuit 33 may be configured to receive DC power from laser source 20 upon as well as following coupling thereto. DC circuit 33 may be configured to store the product key as well as the product key database(s) described herein. DC circuit 33 as well as access control circuit 41 may be configured to store as well as process product key authentication algorithm as well as product key comparison algorithm as described herein. DC circuit 33 as well as access control circuit 41 may be configured to store as well as record (e.g., using mutable data storage) information related to testing, modification, as well as usage of laser delivery device 30, such as date and time of connection to and disconnection from laser source 20, identity of laser source 20 for each connection, durations of laser on as well as laser off, laser output energy levels, event logs, error logs, testing/modification logs, and others.

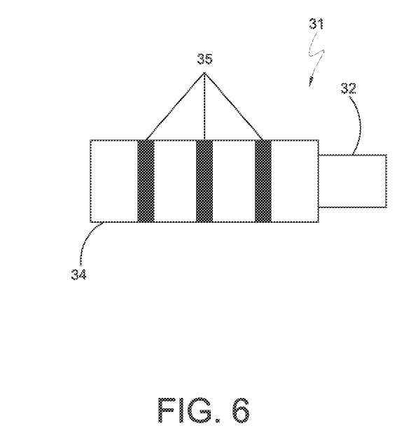

In certain embodiments, as illustrated in FIG. 6, connector 31 may have an outer surface 34 or a portion thereof that may be substantially cylindrical. One or more (e.g., 2, 3, 4 or more) electrical contact(s) 35 (e.g., bands, strips) may be concentrically as well as coaxially disposed along outer surface 34 of connector 31. Contact 35 may be formed of electrically conductive materials, such as metals (e.g., gold, silver), electrically conductive polymeric compositions, or electrically conductive ceramic materials. Contact 35 may be preformed (e.g., tubes, caps, rings), printed (e.g., using electrically conductive paint or ink), embedded, wrapped, wound, deposited (e.g., chemical or physical vapor deposition), plated, or otherwise implemented on outer surface 34. Contact 35 may be flush with outer surface 34. Contact 35 may be circular or a portion of a circle. Any two adjacent contacts 55 may be electrically insulated with insulating materials known in the art, such as polyimides and polyamides. Three or more contacts 55 may be adequate to allow bi-directional or two-way communication between DC circuit 33 of connector 31 and laser source 20 or access control circuit 41 therein. Alternative to the illustration in FIG. 6, contacts may form one of distal and proximal ends of outer surface 34, or both.

To accommodate the contact 35 of FIG. 6, the corresponding electrical contacts in the laser source may be arranged concentrically as well as coaxially with respect to the laser output coupler configured for coupling to the optical connector of the laser delivery device. Such corresponding contacts may be in the form of rings, clamps, pins, blades, protrusions, or other configurations known to one of ordinary skill in the art.

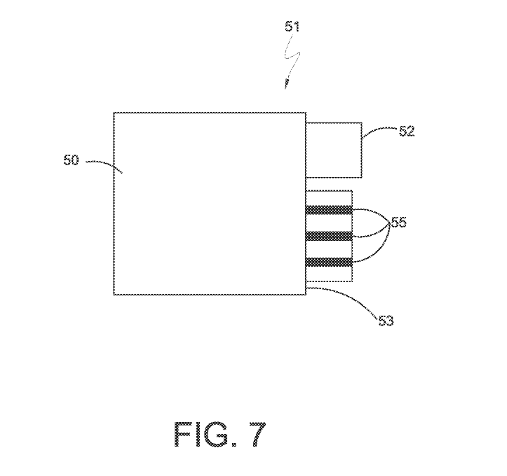

In certain embodiments, as illustrated in FIG. 7, connector 51 may have a body 50 that contains DC circuit 33 (not shown). Optical connector 52 may or may be not a component of DC circuit 33. Optical connector 52 may be substantially cylindrical, or tubular with a polygonal cross-section (e.g., triangular, square, rectangular, rhombus, pentagonal, hexagonal, as well as octagonal). One or more contact substrate(s) 53 (e.g., printed circuit board) may be disposed radially adjacent to optical connector 52. On contact substrate 53 may be disposed one or more (e.g., 2, 3, 4 or more) electrical contact(s) SS (e.g., strips, dots, bands). Contact 55 may likewise be printed, embedded, deposited, plated, or otherwise implemented on substrate 53. Contact 55, when in strip form, may be oriented to be parallel to a longitudinal axis of optical connector 52.

To accommodate the contact 55 of FIG. 7, the corresponding electrical contacts in the laser source may be arranged radially adjacent to the laser output coupler configured for coupling to the optical connector of the laser delivery device. Such corresponding contacts may be in the form of clamps, slots, recesses, or other configurations known to one of ordinary skill in the art.

In other embodiments, the one or more electrical contact(s) implemented on the laser delivery device may be sockets for receiving pins or blades. In other embodiments, the one or more electrical contact(s) may be longitudinal strips or bands extending along the longitudinal axis of the laser delivery device.

The contacts that are disposed on the laser delivery device (e.g., on the proximal connector thereof, as described herein, may be exposed electrical contacts on an outer surface of the laser delivery device. The contacts may be in electrical connection with the DC circuit disposed within the laser delivery device. The contacts may be configured to make electrical contact with contacts implemented in or on laser source, so that DC circuit in the laser delivery device may be electrically coupled to access control circuit within laser source. Upon making such contact, unidirectional (one-way) or bi-directional (two-way) communication may be enabled between DC circuit in laser delivery device and access control circuit in laser source. DC circuit having data storage therein may be able to send stored information about the laser delivery device to laser source or access control circuit therein, such as but not limited to: the product key, supplier identify, model number, device type, serial number, date of manufacturer, expiration date, prior usage history (e.g., number of connects and disconnects, date and time thereof, durations between connects and disconnects, usage parameters and conditions, event logs, error logs, testing logs, modification logs, identity of laser sources connected), and other information general or unique to the laser delivery device. Laser source or access control circuit therein may have a mutable data storage and a processor to record all the information received from laser delivery device, or only selected data if so programmed in the processor. When bi-directional communication is enabled, laser source or access control circuit therein may send certain stored information about laser source (e.g., product key of the laser source) to DC circuit in the laser delivery device. If new procedure(s) is being carried out using the laser delivery device, new information of date and time of connect(s) and disconnect(s) and durations there between, as well as parameters and conditions of the new procedure(s) may be transmitted to DC circuit in the laser delivery device. Laser delivery device or DC circuit therein may have a mutable data storage and optionally a processor, to record all the new information received from laser source, or only selected data if so programmed in the processor.

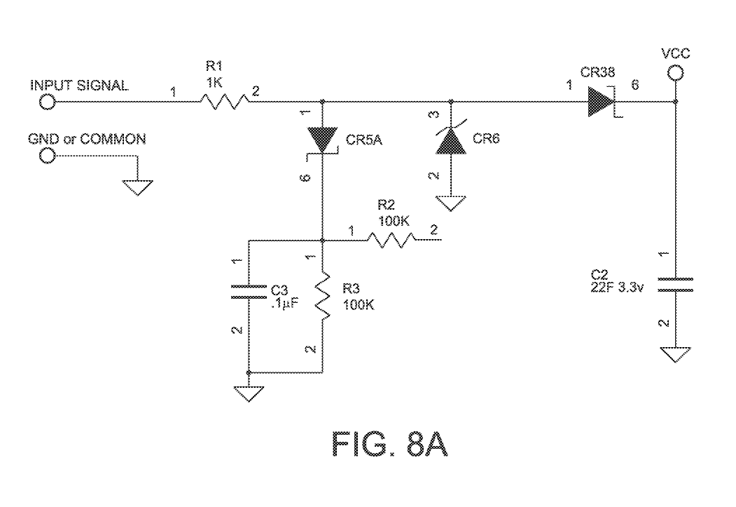

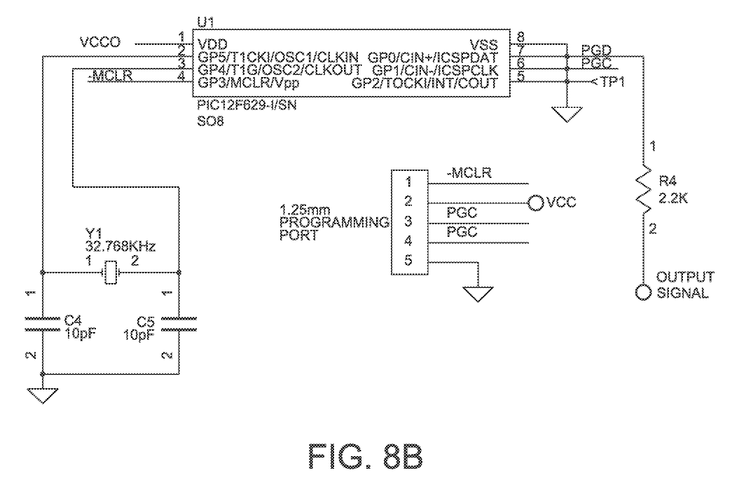

DC circuit in the laser delivery device may be configured to carry out the one or more desired functions described herein. FIG. 8 illustrates an exemplary schematic of a suitable DC circuit. This schematic includes a DC power storage (capacitor C2), a processor with on-board mutable memory (microcontroller U1), and a time keeper (crystal oscillator Y1). When this DC circuit is electrically coupled to a laser source, DC power coming from the laser source through input signal line charges capacitor C2, which powers the DC circuit. When C2 is fully charged, a signal is sent along the master clear line (MCLR) to initiate/activate processor U1. Processor U1 follows the program pre-loaded onto the on-board memory (e.g., by manufacturer through data port P1) to send device information such as its product key through output signal line to the laser source or the access control circuit therein. Laser source may encode information (e.g., laser source product key, information about new usage) in the form of DC pulses of various combinations of frequencies, durations, intervals, as well as waveforms, and send such data-encoded pulses through the same input signal line into the DC circuit. Processor U1 may be able to use pre-loaded decoding algorithm to decode the pulses and record the received information on the on-board mutable data storage. As such, this DC circuit illustrated in FIG. 8 enables unidirectional and bi-directional communication between laser delivery device and laser source.

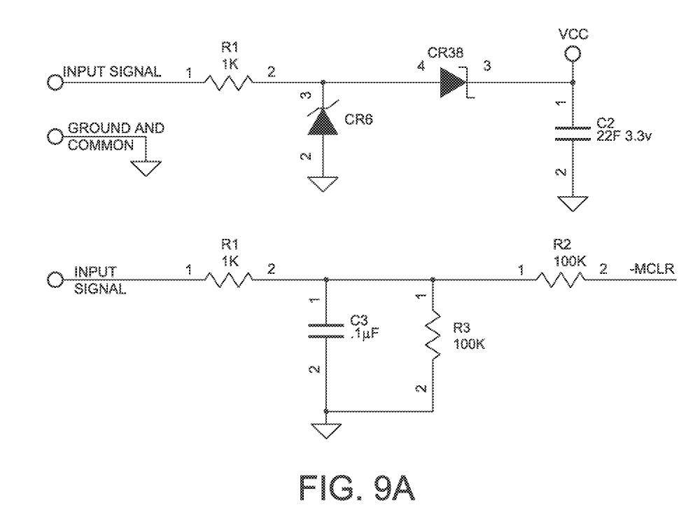

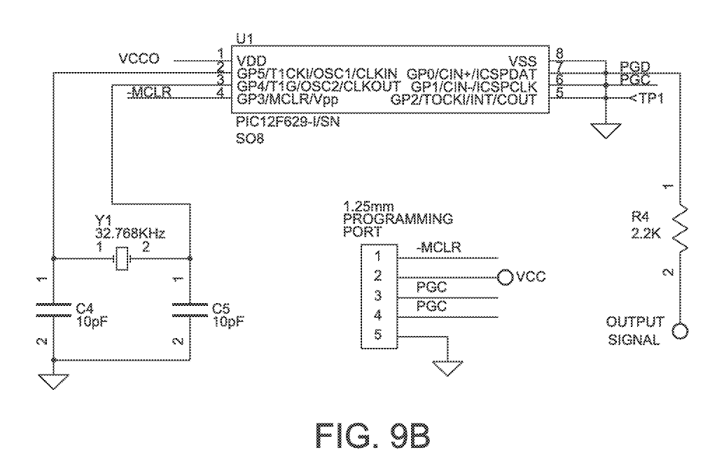

FIG. 9 illustrates another exemplary schematic of a suitable DC circuit. This schematic differs from the one illustrated in FIG. 8 in that two separate lines are used to charge the DC power storage (through +VCC line) and send data from laser source to the DC circuit (through input signal line). As such, there is no need to encode the data transmitted from laser source to the DC circuit in DC pulses. Similar to the DC circuit illustrated in FIG. 8, this DC circuit illustrated in FIG. 9 enables unidirectional and bi-directional communication between laser delivery device and laser source.

Alternative to capacitors and batteries, photovoltaic cells may be suitable DC power storage for the DC circuits used in the laser delivery circuits described herein. The photovoltaic cell may be implemented on an exposed outer surface of the laser delivery device (e.g., on the proximal connectors so that it can be easily and quickly charged in clinical settings where the ambient light is sufficiently bright. Alternatively, the photovoltaic cell may be positioned along a portion of an optical fiber in the laser deliver device and charged by light leaked out of the optical fiber. To enhance the light leakage, a bend such as an S shape may be formed along a segment of the optical fiber within the proximal connector. The photovoltaic cell may be positioned proximate to the bend to receive the leaked light and be charged. Non-limiting examples of photovoltaic cells include: silicon wafer-based cells, epitaxial photovoltaic cells (e.g., amorphous silicon, polycrystalline silicon, micro-crystalline silicon, cadmium telluride, copper indium selenide/sulfide, gallium arsenide), photo electrochemical cells, polymer cells, nanocrystalline cells, dye-sensitized cells, sliver cells.

It is noted that FIGS. 1 and 5 illustrate the body 17 of laser delivery device carrying optical fiber(s) as extending distally from the proximal connector in a direction parallel to, or superimposes with, a longitudinal axis of the proximal connector. In alternative embodiments, the body 17 may turn an angle with respect to the longitudinal axis of the proximal connector, such as between 30 degrees and 90 degrees.

In certain embodiments, the DC circuits described herein may be implemented other than in the proximal connector of the laser delivery device. The DC circuit may be implemented in a separate hardware piece (e.g., a dongle) that is included within the kit that includes the laser delivery device, or supplied separately from the kit without having to be sterilized. Any and all features and functions described herein in associate with the DC circuit and the proximal connector of the laser delivery device may be implemented independently or in combinations thereof in the separate hardware piece. The separate hardware piece containing the DC circuit may be coupled to a communication interface in or on the laser source. Non-limiting examples of such communication interfaces may be serial or parallel, and include: LEMO connectors (e.g., those with 3, 4 or more pins), 3-, 4- or more wire jacks, Ethernet, FireWire, USB, 25-pin D-type connector, DE-9 connector, 9-pin D-subminiature connector, 3-pin through 9-pin standard mini-DIN connectors, 7-pin laptop video connector, 9-pin Apple GeoPort connector, 10-pin connectors, 5-pin MIDI connector, JVC mini-DIN 8, PS/2 connector, DIN connectors, and printer ports. In certain embodiments, the interlock 25 as depicted in FIG. 2 may be modified to implement therein a DC circuit as described herein. Alternatively, a communication interface (e.g., a data port) may be implemented on any part (e.g., front panel, side panel, as well as top panel) of the laser source to accommodate the coupling of the separate hardware piece with the DC circuit therein. Separate hardware pieces that are configured (through instructions pre-loaded into the data storage on the DC circuit) to allow single use may be included in the kit that further include the laser delivery device. Separate hardware pieces that are configured to allow multiple uses (e.g., 5 or more, 10 or more, 100 or more, 1,000 or more) may be supplied separately from the laser delivery device kit.

The proximal connectors (with or without the body of the laser delivery device coupled thereto) or the separate hardware pieces disclosed herein may further be configured as software upgrade tools for laser source software upgrade in the field by sale people or service technicians. The laser source may be configured (through programs pre-loaded onto a data storage therein) to query the software version on the proximal connector or the separate hardware piece, compare that with the on-board software version, decide whether or not to upgrade, and proceed to upgrade when deemed desirable. The proximal connectors (with or without the body of the laser delivery device coupled thereto) or the separate hardware pieces may further be configured as laser source service and maintenance tools. They can be used to download stored information in the laser source (e.g., event logs, error logs) and be sent back to supplier or manufacturer for diagnosis or calibration, without having to ship the expensive and bulky laser source.

Throughout the present disclosure in its entirety, any and all of the one, two, or more features disclosed herein following the terms “examples” and “embodiments” may be practiced in any combinations of two, three, or more thereof, whenever and wherever appropriate as understood by one of ordinary skill in the art. Some of these examples are themselves sufficient for practice without being combined with any other features, as understood by one of ordinary skill in the art. Throughout the present disclosure in its entirety, any and all of the descriptions following the term “example” are for illustration only, without limiting the scope of any of the referenced terms or phrases either within the context or outside the context of such descriptions.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Thus, for example, the reference to a microparticle is a reference to one such microparticle or a plurality of such microparticles, including equivalents thereof known to one skilled in the art. Also, as used herein and in the claims, the terms “at least one” and “one or more” have the same meaning and include one, two, three or more. The following terms, unless otherwise indicated, shall be understood to have the following meanings when used in the context of the present disclosure.

Other than in the operating examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

Notwithstanding that the numerical ranges, and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values can be used.

Examples provided herein, including those following “such as” and “e.g.,” are considered as illustrative only of various aspects and features of the present disclosure and embodiments thereof, without limiting the scope of any of the referenced terms or phrases either within the context or outside the context of such descriptions. Any suitable equivalents, alternatives, and modifications thereof (including materials, substances, constructions, compositions, formulations, means, methods, conditions, etc.) known or available to one skilled in the art can be used or carried out in place of or in combination with those disclosed herein, and are considered to fall within the scope of the present disclosure. Throughout the present disclosure in its entirety, any and all of the one, two, or more features and aspects disclosed herein, explicitly or implicitly, following terms “example”, “examples”, “such as”, “e.g.”, and the likes thereof may be practiced in any combinations of two, three, or more thereof (including their equivalents, alternatives, and modifications), whenever and wherever appropriate as understood by one of ordinary skill in the art. Some of these examples are themselves sufficient for practice singly (including their equivalents, alternatives, and modifications) without being combined with any other features, as understood by one of ordinary skill in the art. Therefore, specific details disclosed herein are not to be interpreted as limiting, but merely as a basis for the claims and as a representative basis for teaching one skilled in the art to variously employ aspects and features of the present disclosure in virtually any appropriate manner.

1. A kit comprising a treatment delivery device coupled to an electromagnetically readable product key that is detachable from said treatment delivery device, wherein said treatment delivery device is configured for coupling with a treatment source, and said product key is electromagnetically readable by said treatment source.

2. The kit of claim 1 wherein said key is physically separate from said treatment delivery device.

3. The kit as in any one of the preceding claims, in which said treatment delivery device is a laser delivery device.

4. An apparatus comprising a treatment delivery device coupled to a circuit powered by direct current, wherein the circuit is coupled to a direct current power storage and a data storage, and said data storage comprises an electromagnetically readable product key unique to said treatment delivery device.

5. An apparatus comprising: a laser delivery device coupled to a direct current circuit wherein said direct current circuit is coupled to a mutable data storage and said mutable data storage comprises an electromagnetically readable product key unique to said laser delivery device.

6. An apparatus comprising a hardware piece coupled to a direct current circuit wherein said direct current circuit is coupled to a direct current power storage and a mutable data storage, and said mutable data storage comprises an algorithm for limiting usage of a treatment delivery device.

7. The apparatus of claim 6 wherein usage is limited by at least one of time of usage and number of usages.

8. The apparatus of claim 6, wherein the hardware piece is a door interlock.
